(19) United States
(12) Patent Application Publication
(10) Pub. No.: US 2020/0392134 A1
(43) Pub. Date: Dec. 17, 2020

(54) METHOD FOR PURIFYING 2-[[2-[[[3-(4-CHLOROPHENYL)-8-METHYL-8-AZABICYCLO-[3.2.1]-OCT-2-YL]METHYL](2-MERCAPTOETHYL)AMINO]ETHYL]AMINO]ETHANETHIOL-[1R-(EXO-EXO)]TRIHYDROCHLORIDE

(71) Applicant: INSTITUTE OF NUCLEAR ENERGY RESEARCH, ATOMIC ENERGY COUNCIL, EXECUTIVE YUAN, TAOYUAN CITY (TW)

(72) Inventors: SHOW-WEN LIU, TAOYUAN CITY (TW); CHENG-FANG HSU, TAOYUAN CITY (TW); WEI-HSI CHEN, TAOYUAN CITY (TW); YU CHANG, TAOYUAN CITY (TW)

(21) Appl. No.: 15/930,275

(22) Filed: May 12, 2020

(30) Foreign Application Priority Data
Jun. 11, 2019 (TW) 108120150

(51) Int. Cl. C07D 471/08

(52) U.S. Cl. CPC C07D 471/08

(57) ABSTRACT

A method for purifying 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)]-hydrochloride is revealed. After medium pressure liquid chromatography and subsequent acid treatment, 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)]-hydrochloride with high purity is obtained. The method for purifying can solve the problem that the product purity is not up to the standard for radiopharmaceuticals.

### BACKGROUND OF THE INVENTION

### Field of the Invention

The present invention relates to a method for purifying a precursor of an imaging agent, especially to a method for purifying 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)]trihydrochloride.

### Description of Related Art

According to Florida hospital statistics, at least one million people in US suffer from Parkinson's disease. There are about sixty thousand people who get diagnosed with Parkinson's disease annually and approximately seven million to ten million people live with this disease on a global scale. Not only the health insurance premium but also the average medication costs for each patient are huge.

As stated by the official health statistics, the diagnosis and treatment of the disease have been estimated to incur cost of over 25 billion in the US each year. The yearly medication cost for treatment of each patient is about $2, 500 and even up to $10,000 with surgery. Thus there is an urgent need to diagnose and treat Parkinson's disease in its early stage.

99mTc-labeled dopamine transporter imaging agents have been developed and used in radiopharmaceuticals. Among related literature, most attentions have been given to [99mTc]TRODAT-1 reported by Dr. Hank F. Kung and his colleagues in University of Pennsylvania. After a series of tests, it has been approved that [99mTc]TRODAT-1 is a potential imaging agent in clinical, worth further development in the country, and successfully applied to scanning/imaging of the brain's basal nuclei. Thus this novel imaging agent improves convenience and accessibility of brain scans of patients with Parkinson's disease.

2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)]trihydrochloride (abbreviated as TRODAT-1·3HCl)) is one of the active pharmaceutical ingredients (API) of 99mTc-labeled dopamine transporter imaging agents ([99mTc]TRODAT-1 imaging agent) used for evaluation of brain function in nuclear medicine imaging procedures.

However, the production processes of TRODAT-1·3HCl are complicated and up to 11 steps. In the last step, thiol protecting groups in 2-[[2-[[[3-(4-Chlorophenyl)-8-methyl-8-azabicyclo[3.2.1]-oct-2-yl]methyl] [[S-(4-methoxybenzyl)thio]ethyl]amino]ethyl]amino]-S-(4-methoxybenzyl) ethanethiol-[1R-(exo-exo)] are removed to get the final product TRODAT-1·3HCl.

There are two methods to remove thiol protecting group. One way is taking 2-[[2-[[[3-(4-Chlorophenyl)-8-methyl-8-azabicyclo[3.2.1]-oct-2-yl]methyl] [[S-(4-methoxybenzyl)thio]ethyl]amino]ethyl]amino]-S-(4-methoxybenzyl) ethanethiol-[1R-(exo-exo)] to react with Mercury(II) acetate and then hydrogen sulfide is introduced. The other way is to dissolve 2-[[2-[[[3-(4-Chlorophenyl)-8-methyl-8-azabicyclo[3.2.1]-oct-2-yl]methyl] [[S-(4-methoxybenzyl)thio]ethyl]-amino]ethyl]amino]-S-(4-methoxybenzyl) ethanethiol-[1R-(exo-exo)] in trifluoroacetic acid and then add methoxybenzene and methanesulfonic acid. Both methods can get the final product smoothly. The former can get the product with higher purity (95%) but has the shortcoming of residual mercury being detected. The later uses mercury free process yet the purity of the product is often about 90% of the API requirement.

As an important API of radiopharmaceuticals, the purity of TRODAT-1·3HCl should be larger than 98% to meet requirements for the purity of radiopharmaceuticals. During preparation process, TRODAT-1·3HCl is easy to be oxidized to form S—S bonds under neutral or alkaline condition owing to dithiol groups in TRODAT-1·3HCl and hence unable to generate S—M (M═Tc or Re) bonds. Thus the oxidized TRODAT-1·3HCl can't be used for preparation of dopamine transporter imaging agents. The difficulty in preparation and high cost of preparation leads to limits on applications of TRODAT-1·3HCl.

Thus there is room for improvement and there is a need to provide a novel method for preparing TRODAT-1·3HCl, by which the problems caused by conventional methods such as difficulty in preparation, high cost of preparation and purity not up to the standard for radiopharmaceuticals can be solved. Thus TRODAT-1·3HCl can be utilized for early diagnosis of the Parkinson's disease more effectively and efficiently.

### SUMMARY OF THE INVENTION

Therefore it is a primary object of the present invention to provide a method for purifying 2-[[2-[[[3-(4-Chlorophenyl)-8-methyl-8-azabicyclo[3.2.1]-oct-2-yl]methyl]-(2-mercaptoethyl)amino]ethyl]amino]ethanethiol-[1R-(exo-exo)]trihydrochloride in which firstly a mixed solution of precursors of dopamine transporter imaging agents is separated by column chromatography to get 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)] trifluoroacetate (abbreviated as TRODAT-1·3CF3CO2H) and then TRODAT-1·3CF3CO2H is treated with hydrochloric acid to get 2-[[2-[[[3-(4-chlorophenyl)-8-methyl-8-azabicyclo[3.2.1]-oct-2-yl]methyl](2-mercaptoethyl)amino]ethyl]amino]ethanethiol-[1R-(exo-exo)]trihydrochloride (abbreviated as TRODAT-1·3HCl) with high purity over 98% so as to solve the problems of the conventional methods such as difficulty in preparation, high cost of preparation, purity not up to the standard for radiopharmaceuticals, etc.

In order to achieve the above object, a method for purifying 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)]trihydrochloride according to the present invention includes the steps of: taking a mixed solution of precursors of dopamine transporter imaging agents to perform column chromatography and get a product; and then taking the product and hydrochloric acid to carry out a substitution reaction and get 2-[[2-[[[3-(4-chlorophenyl)-8-methyl-8-azabicyclo[3.2.1]-oct-2-yl]-methyl](2-mercaptoethyl)amino]ethyl]amino]ethanethiol-[1R-(exo-exo)]trihydrochloride. The mixed solution of precursors of the dopamine transporter imaging agent includes the product and a plurality of impurities.

Preferably, in the method for purifying 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)]trihydrochloride, the impurities include a disulfide bond, a cleavable structure, a structure with uncut thiol protecting group, and a dimer structure.

Preferably, in the step of taking a mixed solution of precursors of dopamine transporter imaging agents to perform column chromatography and get a product, the column chromatography is run by a medium pressure liquid chromatography instrument.

Preferably, in the step of taking a mixed solution of precursors of dopamine transporter imaging agents to perform column chromatography and get a product, the product is 2-[[2-[[[3-(4-Chlorophenyl)-8-methyl-8-azabicyclo[3.2.1]-oct-2-yl]methyl]-(2-mercaptoethyl)amino]ethyl]amino]ethanethiol-[1R-(exo-exo)]-trifluoroacetate.

Preferably, in the step of taking the product and hydrochloric acid to carry out a substitution reaction and get 2-[[2-[[[3-(4-chlorophenyl)-8-methyl-8-azabicyclo [3.2.1]-oct-2-yl]methyl](2-mercaptoethyl)amino]ethyl]amino]ethanethiol-[1R-(exo-exo)]hydrochloride, a trifluoroacetic acid of the 2-[[2-[[[3-(4-Chlorophenyl)-8-methyl-8-azabicyclo[3.2.1]-oct-2-yl]methyl](2-mercaptoethyl)amino]ethyl]amino]-ethanethiol-[1R-(exo-exo)] trifluoroacetate is replaced by the hydrochloric acid in the substitution reaction.

Preferably, the step of taking the product and hydrochloric acid to carry out a substitution reaction further includes the steps of carrying out a concentration process and carrying out a drying process.

Preferably, in the step of carrying out a concentration process, the concentration process is carried out at a lower pressure.

Preferably, in the step of carrying out a drying process, the drying process is run by a freeze dryer.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a flow chart showing steps of an embodiment according to the present invention;

FIG. 2 shows chemical structure of 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)]trihydrochloride according to the present invention;

FIG. 3 shows chemical structure of impurities of an embodiment according to the present invention;

FIG. 4 is ¹H-NMR spectrum of 2-[[2-[[[3-(4-Chlorophenyl)-8-methyl-8-azabicyclo-[3.2.1]-oct-2-yl]methyl](2-mercaptoethyl)amino]ethyl]amino]ethanethiol-[1R-(exo-exo)]trifluoroacetate according to the present invention;

FIG. 5 is ¹H-NMR spectrum of 2-[[2-[[[3-(4-Chlorophenyl)-8-methyl-8-azabicyclo [3.2.1]-oct-2-yl]methyl](2-mercaptoethyl)amino]ethyl]amino]ethanethiol-[1R-(exo-exo)]trihydrochloride according to the present invention;

FIG. 6 is a chromatograph showing purity of 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)]trihydrochloride according to the present invention.

### DETAILED DESCRIPTION OF THE PREFERRED EMBODIMENT

The present invention provides a method for purifying 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)]trihydrochloride that overcomes the shortcomings of the conventional techniques such as difficulty in preparation, high cost of preparation and purity not up to the standard for radiopharmaceuticals.

The features, structure and method of the present invention with details are further described in the following embodiments.

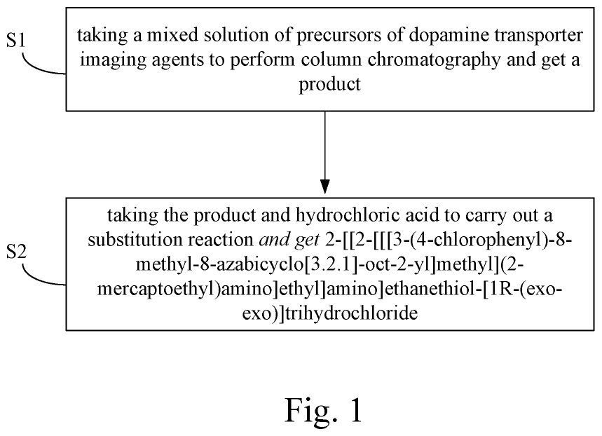

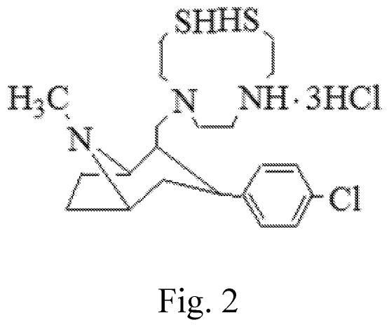

Refer to FIG. 1, a flow chart showing steps of an embodiment is revealed. As shown in FIG. 1, a method for purifying 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)]trihydrochloride (whose chemical structure is shown in FIG. 2) includes the following steps.
S1: taking a mixed solution of precursors of dopamine transporter imaging agents to perform column chromatography and get a product; and
S2: taking the product and hydrochloric acid to carry out a substitution reaction and get 2-[[2-[[[3-(4-chlorophenyl)-8-methyl-8-azabicyclo[3.2.1]-oct-2-yl]methyl](2-mercaptoethyl)amino]ethyl]amino]ethanethiol-[1R-(exo-exo)]trihydrochloride.

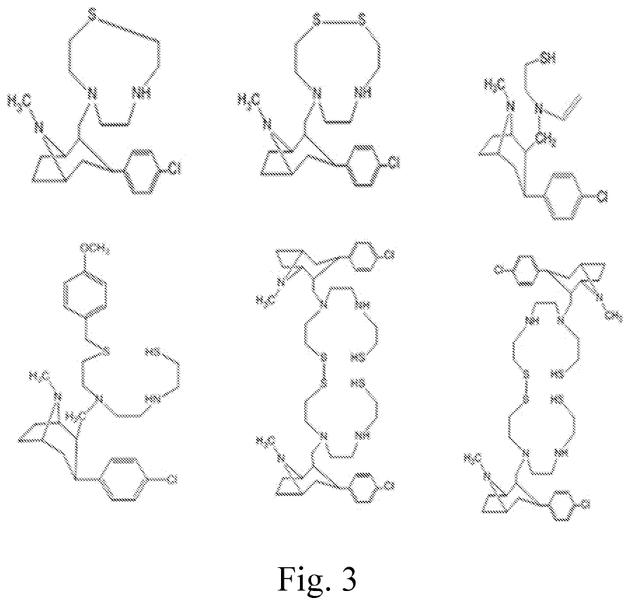

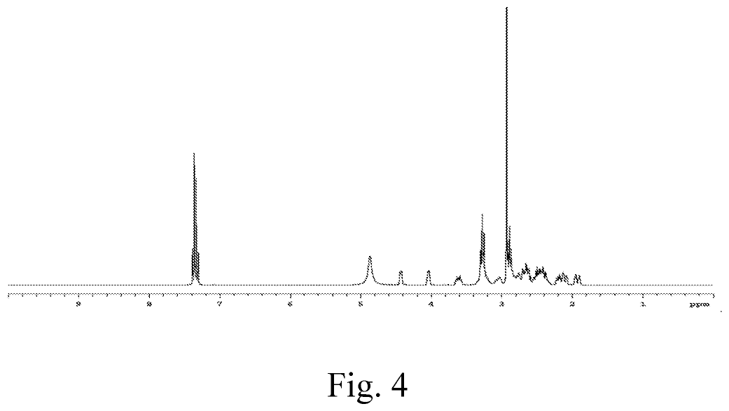

As shown in the step S1, the mixed solution of precursors of dopamine transporter imaging agents includes a plurality of impurities which include a disulfide bond, a cleavable structure, a structure with uncut thiol protecting groups, and a dimer structure. As shown in FIG. 3, there are two disulfide bonds, one cleavable structure, one structure with uncut thiol protecting groups, and two dimers. The column chromatography is run by a medium pressure liquid chromatography instrument under the following conditions: mobile phase: 50/50 methanol/water containing 0.1% trifluoroacetic acid; column: RedSep HP C18 (50 g, gold, sample amount 50 mg˜1 g); flow rate: 25 mL/min; UV detection wavelength: 210 nm; analysis time: 20 min; and injection amount: 0.5 g sample/1 mL methanol/each time. Different concentrations of methanol are used as eluent in column chromatography and the column is eluted with 10% methanol→50% methanol→100% methanol→10% methanol. The total elution time is 20 minutes. Later collect the respective signal tubes and the product is dried by a freeze dryer. Measure the ¹H-NMR spectrum of the product to determine the molecular structure of the product according to absorbance peaks in the ¹H-NMR spectrum, as shown in FIG. 4. The product is 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)] trifluoroacetate.

Then as shown in the step S2, taking the product (2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)] trifluoroacetate) and hydrochloric acid to carry out a substitution reaction. The hydrochloric acid is concentrated hydrochloric acid and the amount of the hydrochloric acid added is three times of the number of moles of the product. The number of moles of the product*3*36.5/38%=the amount of the hydrochloric acid required (unit: milliliter). Before carrying out the substitution reaction, dissolve the product in anhydrous methanol (20 ml). Then stir the solution at room temperature for 10 minutes after adding hydrochloric acid. In the substitution reaction, a trifluoroacetic acid of the 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)] trifluoroacetate is replaced by the hydrochloric acid.

The step S2 of taking the product and the hydrochloric acid to carry out a substitution reaction further includes the steps of:
S21: carrying out a concentration process, and
S22: carrying out a drying process.

As shown in the step S21, a concentration process is run at a lower pressure and the operating temperature is lower than 35 degrees Celsius (° C.).

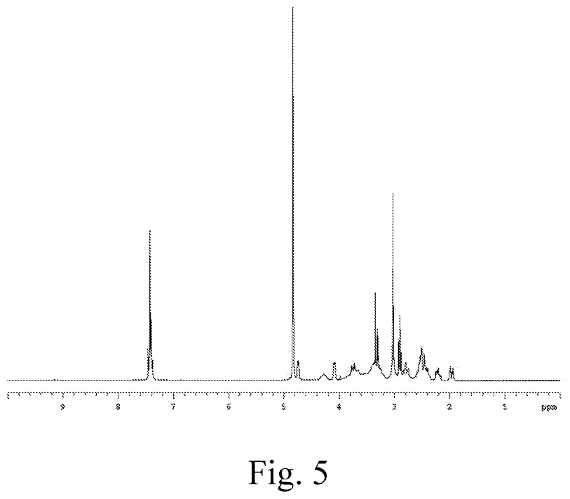

Lastly, as shown in the step S22, dry the product at high vacuum conditions by a freeze dryer and then measure the 1H-NMR spectrum of the product. Based on the absorbance peaks in the 1H-NMR spectrum (as shown in FIG. 5), the product is 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)]trihydrochloride (abbreviated as TRODAT-1·3HCl).

The spectral data of TRODAT-1·3HCl is as following:

¹H NMR (CD₃OD) δ 7.45 (m, 4 H, C₆H₄), 4.75 (m, 1 H), 4.32 (m, 1 H), 4.12 (br, 1 H), 3.20-3.98 (m, 14 H), 3.05 (s, 3 H, NCH₃), 2.73-3.05 (m, 3 H), 2.31-2.70 (m, 5 H), 2.24 (m, 1 H), 2.00 (d, br, 1 H)° ¹³C NMR (CD₃OD) δ 138.69, 134.08, 130.68, and 130.45 (C₆H₄), 66.94 (CH), 65.69 (CH), 51.97 (CH₂), 43.01 (CH₂), 41.29 (CH), 40.32 (NCH₃), 34.07 (CH), 31.62 (CH₂), 26.07 (CH₂), 24.31 (CH₂), 21.52 (CH₂)° MS m/z 427 (M⁺), 394 (M⁺-SH), 361 (M⁺-2 (SH)).

Moreover, use High Performance Liquid Chromatography (HPLC) instrument to verity the purity of TRODAT-1·3HCl. The column chromatography is run under the following conditions: mobile phase: 50/50 methanol/water containing 0.1% trifluoroacetic acid; column Chromolithic C18 (4.6*100 mm); flow rate: 0.5 mL/min; UV detection wavelength: 210 nm; and analysis time: 30 min.

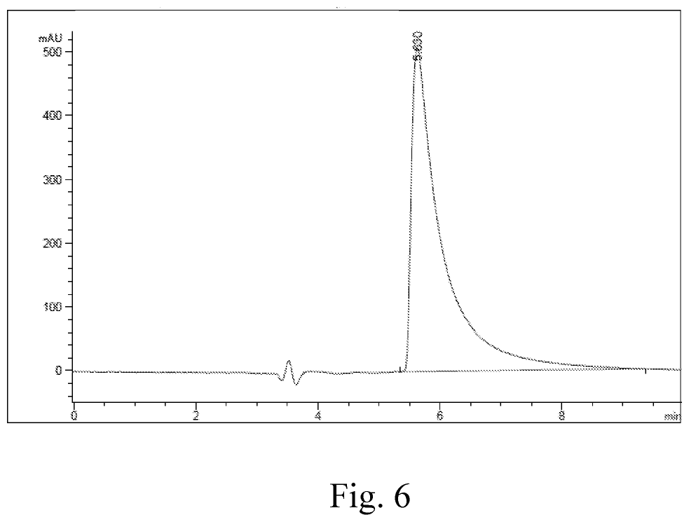

The purity is 100% (as the result shown in FIG. 6). The HPLC analysis data is shown in the following table 1. The 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)]trihydrochloride produced by the present method meets the requirements for the purity of radiopharmaceuticals.

TABLE 1
HPLC analysis data

| | | | Width | Area | | |
|---|---|---|---|---|---|---|
| Peak | RetTime(min) | Type | (min) | (nAU*s) | Height (mAU) | Purity (%) |
| 1 | 5.603 | BB | 0.4844 | 1.77735e4 | 507.77493 | 100.0000 |
| Totals | | | | 1.77735e4 | 507.77493 | |

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

What is claimed is:

1. A method for purifying 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)]trihydrochloride comprising the steps of:
taking a mixed solution of precursors of dopamine transporter imaging agents to perform column chromatography and get a product; and
taking the product and hydrochloric acid to carry out a substitution reaction and get 2-[[2-[[[3-(4-chlorophenyl)-8-methyl-8-azabicyclo [3.2.1]-oct-2-yl]methyl](2-mercaptoethyl)amino]ethyl]amino]ethanethiol-[1R-(exo-exo)]trihydrochloride;
wherein the mixed solution of precursors of dopamine transporter imaging agents includes the product and a plurality of impurities.

2. The method as claimed in claim 1, wherein the impurities include a disulfide bond, a cleavable structure, a structure with uncut thiol protecting group, and a dimer structure.

3. The method as claimed in claim 1, wherein in the step of taking a mixed solution of precursors of dopamine transporter imaging agents to perform column chromatography and get a product, the column chromatography is run by a medium pressure liquid chromatography instrument.

4. The method as claimed in claim 1, wherein in the step of taking a mixed solution of precursors of dopamine transporter imaging agents to perform column chromatography and get a product, the product is 2-[[2-[[[3-(4-Chlorophenyl)-8-methyl-8-azabicyclo[3.2.1]-oct-2-yl]methyl](2-mercaptoethyl)amino]ethyl]amino]ethanethiol-[1R-(exo-exo)] trifluoroacetate.

5. The method as claimed in claim 4, wherein in the step of taking the product and hydrochloric acid to carry out a substitution reaction and get 2-[[2-[[[3-(4-chlorophenyl)-8-methyl-8-azabicyclo[3.2.1]-oct-2-yl]methyl](2-mercaptoethyl)amino]ethyl]amino]ethanethiol-[1R-(exo-exo)]trihydrochloride, a trifluoroacetic acid of the 2-[[2-[[[3-(4-Chlorophenyl)-8-methyl-8-azabicyclo-[3.2.1]-oct-2-yl]methyl](2-mercaptoethyl)amino]ethyl]amino]ethanethiol-[1R-(exo-exo)] trifluoroacetate is replaced by the hydrochloric acid in the substitution reaction.

6. The method as claimed in claim 1, wherein the step of taking the product and hydrochloric acid to carry out a substitution reaction further includes the steps of:
carrying out a concentration process, and
carrying out a drying process.

7. The method as claimed in claim 6, wherein in the step of carrying out a concentration process, the concentration process is carried out at a lower pressure.

8. The method as claimed in claim 6, wherein in the step of carrying out a drying process, the drying process is carried out by a freeze dryer.
